(19) United States
(12) Patent Application Publication
(10) Pub. No.: US 2023/0079389 A1
(43) Pub. Date: Mar. 16, 2023

(54) COMPOSITION FOR USE IN PREVENTION OR TREATMENT OF OESOPHAGEAL DISEASES LINKED TO EPITHELIAL BARRIER DEFECTS

(71) Applicant: THELIAL B.V., Wageningen (NL)

(72) Inventors: Luciana VIERIA DE MORAES, Wageningen (NL); Richard John HAMPSON, Wageningen (NL)

(21) Appl. No.: 17/802,183

(22) PCT Filed: Feb. 5, 2021
(86) PCT No.: PCT/EP2021/052808

§ 371 Date: Aug. 25, 2022

(30) Foreign Application Priority Data
Feb. 27, 2020 (NL) 2025009

(51) Int. Cl. A61K 31/352; A61K 31/4375; A61K 31/734; A61P 1/04

(52) U.S. Cl. CPC A61K 31/352; A61K 31/4375; A61K 31/734; A61P 1/04

(57) ABSTRACT

A composition comprising quercetin (2-(3,4-dihydroxyphenyl)-3,5,7-trihydroxychromen-4-one) and/or myricetin (3,5,7-Trihydroxy-2-(3,4,5-trihydroxyphenyl)-4H-1-benzopyran-4-one) is used in the prevention or treatment of heartburn, gastroesophageal reflux disease (GERD) or Eosinophilic Esophagitis (EoE). The composition reinforces tight junction strength in the oesophagus wall at low concentrations, restoring healthy barrier function to the oesophagus and resistance to acid. The composition prevents acid from penetrating the oesophagus, irritating underlying nerves and causing pain.

### FIELD OF THE INVENTION

The present disclosure relates to a composition for use in prevention or treatment of heartburn, gastroesophageal reflux disease (GERD) and Eosinophilic Esophagitis (EoE).

### BACKGROUND OF THE DISCLOSURE

Acid reflux, stomach acid entering into the oesophagus tube which connects mouth and stomach, is known by virtually all of us and can cause heartburn. When it is a common occurrence it is termed Gastroesophageal reflux disease (GERD).

GERD sufferers have an oesophagus lining with reduced barrier strength because the connections between barrier cells—formed by tight junctions—are impaired. Therefore, the oesophagus lining is not fully resistant to the rising stomach acid, and as a consequence acid can enter the tissue and irritate underlying nerves.

Heartburn and GERD affects up to 20% of the population and is linked to impaired quality of life and significant health care costs (>15B US$ per year worldwide). Pregnant women have a particularly great need, with up to 80% suffering heartburn and GERD.

Current solutions are not optimal: heartburn and GERD is at the moment most commonly managed by controlling stomach acid, either through chemical neutralization of acid present in the stomach or drug-based suppression of acid production, the latter frequently with proton pump inhibitors (PPIs).

PPIs have in the past been very successful but there are key issues in that (i) they are poorly accepted by pregnant women, as they are reported to put the child at risk of asthma and other avoidable diseases, (ii) 30% of patients in the general population do not respond and (iii) increasingly side effects are reported bringing into doubt their suitability for long-term management of oesophagus health.

EoE is a chronic immune mediated disorder characterised by high counts of a specific immune cell type, eosinophils, in the oesophagus tissue.

EoE is a rare disease, in Europe it affects 50-100 people per 100,000. All age-groups are affected.

Current management of EoE is not ideal, focussing on dietary restrictions and management of immune reactions by long-term usage of corticosteroid drugs such as Budesonide, either off-label or in the targeted formulation of Jorveza (Dr. Falk Pharma).

Dietary management is restrictive and corticosteroids such as Budesonide are reported to have potentially severe side-effects especially in long-term usage. Targeted formulation Jorveza is not approved or tested in paediatric patients.

### SUMMARY OF THE INVENTION

It is an object of the present invention to overcome one or more problems in the prior art, in particular to come to an effective means to prevent and/or treat heartburn, GERD and EoE, preferably with no or little side effects.

To solve the above problems, a composition comprising quercetin (2-(3,4-dihydroxyphenyl)-3,5,7-trihydroxychromen-4-one) and/or myricetin (3,5,7-Trihydroxy-2-(3,4,5-trihydroxyphenyl)-4H-1-benzopyran-4-one) is used in the prevention or treatment of heartburn, gastroesophageal reflux disease and Eosinophilic Esophagitis. Surprisingly a combination of quercetin and myricetin is particularly effective in treatment of heartburn, GERD and EoE. The composition according to the present invention may strengthen oesophagus wall barrier function, e.g. by reinforcing tight junction strength between epithelial cells in the oesophagus wall, thereby restoring healthy barrier function to the oesophagus and epithelial resistance. Tight junction strength between epithelial cells (which form the continuous intercellular barrier) can be measured by in situ determining intraluminal esophageal electrical impedance (e.g. by esophagoscopy), or by taking a biopsy and measuring transepithelial electrical resistance or by using transepithelial permeability marker dyes. In heartburn and GERD the composition prevents acid from penetrating the oesophagus, irritating underlying nerves and causing pain. In EoE the composition prevents allergens from penetrating the oesophagus and triggering an immune response.

### DETAILED DESCRIPTION OF THE INVENTION

Gastro-intestinal reflux disease (GERD) is the same as Gastroesophageal reflux disease or acid reflux. It is a condition wherein stomach contents, in particular stomach acid, rises up into the oesophagus. The stomach acid causes pain, in particular chest pain or heartburn or pyrosis. Other symptoms of GERD are bad breath and tooth decay.

Eosinophilic Esophagitis (EoE) is a chronic immune disorder where allergens from food trigger an exaggerated immune reaction, causing painful inflammation, impossibility to swallow, food compaction, vomiting and other. Often EoE patients have weakened oesophagus epithelia, meaning allergens pass more easily into the underlying tissue, where the aberrant immune response is triggered. As EoE is a rare disease the time from presentation to diagnosis is often prolonged.

A Composition is herein understood as a product of several ingredients. Compositions can be foods, drinks, medicines, etc. In particular, the composition referred to herein can be ingested and/or may be comprised in a medical device.

Quercetin is also known as 5,7,3′,4′-flavon-3-ol, and occurs in nature. In particular in fruits, vegetables, and cereals. Quercetin is a flavonol and is known as an ingredient in foods and drinks and as a dietary supplement. Administered systemically at high dose (such as 500 mg, the same amount as present in e.g. 40 kg of Jonagold apples) may have antioxidant activity and stimulate the immune system.

Myricetin is also known as Cannabiscetin, Myricetol and Myricitin. Myricetin is derived from fruits, vegetables, nuts, and tea. Myricetin is an antioxidant, that protects against some forms of cancer and cardiovascular disease.

The composition comprises ideally amounts of Quercetin typically encountered in foodstuffs. In the case of Quercetin for example the same amount as present in 3 Jonagold apples (530 g) or a single serving 10-15 commercially prepared capers. In line with this, such a low-dose supplement can be used by pregnant women suffering heartburn or GERD and children with EoE; high dosage supplements are not recommended in either of these patient populations.

The composition according to the invention comprises quercetin (2-(3,4-dihydroxyphenyl)-3,5,7-trihydroxychromen-4-one) and/or myricetin (3,5,7-trihydroxy-2-(3,4,5-trihydroxyphenyl)-4H-1-benzopyran-4-one) and may be used for preventing and/or treating heartburn, gastroesophageal reflux disease and/or Eosinophilic Esophagits. Quercetin has been found to be effective in treating and/or preventing heartburn, GERD and/or EoE.

Myricetin has been found to be effective in treating and/or preventing heartburn, GERD and/or EoE as well.

In another aspect of the invention, the composition according to the invention comprises quercetin (2-(3,4-dihydroxyphenyl)-3,5,7-trihydroxychromen-4-one) and myricetin (3,5,7-trihydroxy-2-(3,4,5-trihydroxyphenyl)-4H-1-benzopyran-4-one) and may be used for preventing and/or treating gastroesophageal reflux disease. The combination of quercetin and myricetin has been found to be particularly synergistic in treating and or preventing heartburn, GERD and/or EoE.

The composition according to the invention may also comprise genistein (5,7-dihydro-3-(4-hydroxyphenyl)chromen-4-one) and/or berberine (5,6-dihydro-9,10-dimehtoxybenzo[g]-1,3-benzodioxolo[5,6-a]quinolizinium). Genistein and berberine are examples of flavonoids which reinforce tight junctions but do not modulate Par6 as quercetin and myricetin do.

Having two molecules which both reinforce acting via different routes reinforces their synergy. The following compositions are effective to treat heartburn, GERD and/or EoE: quercetin and genistein; quercetin and berberine; myricetin and genistein; myricetin and berberine; quercetin, myricetin, and genistein; quercetin, myricetin, and berberine; and quercetin, myricetin, genistein, and berberine.

The amount of quercetin in the composition according to the invention may range between 0.01 and 1000 mg, preferably between 0.1 mg and 100 mg, or between 0.1-20 mg, most preferably the amount of quercetin is between 25-50 mg, or about 38 mg. The amount of myricetin may range between 0.01 and 1000 mg, preferably between 0.1 mg and 100 mg, or between 0.1-20 mg, most preferably the amount of myricetin is between 25-50 mg, or about 38 mg. Laboratory and clinical experiments have shown that a dose of 38 mg of quercetin and/or 38 mg of myricetin gave optimal results. Dosages of quercetin and/or myricetin can however range up to 1000 mg. The amounts may be based on a single dose which may be administered (once, twice or three times) daily, or at least 1, 2, 3, 4 times per week.

The amount of genistein in the composition according to the invention may range between 0.01 and 600 mg, preferably between 1 and 100 mg, and most preferably the amount of genistein is about 12 mg. The amount of berberine in the composition may range between 0.01 and 1500 mg, preferably between 0.1 mg and 200 mg, most preferably the amount of berberine is about 1 mg. A dosage of 12 mg genistein and/or 1 mg of berberine is optimal to reinforce barrier tissues in the oesophagus. The amounts may be based on a single dose which may be administered (once or twice) daily, or at least 1, 2, 3, 4 times per week.

Advantageously, the composition according to the invention may be for oral administration and/or present in a solid dosage form, such as a tablet, pill, lozenge, capsule, chewing gum, powder, or granule, preferably the composition according to the invention is present in a lozenge. A solid dosage form allows easy intake of the composition, possibly through orally dispersible lozenge, under the tongue dispersible tablet (or chewing gums). Orally dispersible lozenges, chewing gums and under the tongue dispersible tablets advantageously lengthen the contact time between the composition and the oesophagus, e.g. to at least 1, 2, 3, 4, 5 minutes, thereby further increasing the effectiveness of the composition e.g. in treating and/or preventing heartburn and GERD. Any slow-release formulation that is formulated such that the contact time between the composition and the oesophagus is at least 1, 2, 3, 4, 5 minutes could be used in the present invention, and allows for use of a low dosage as described herein.

A lozenge comprising between 70-98 wt. % isomalt, or up to 95 wt. % or more isomalt, e.g. a 2,5 g compressed lozenge, has an excellent in-mouth dissolution time of more that 5 minutes.

The composition according to the invention may also be present in a liquid dosage form, such as a drink or syrup. This liquid dosage form may be ready to consume or may be a dosage to be prepared by the user. A liquid dosage form allows the entire oesophagus barrier to come into contact with the composition, thereby effectively reinforcing virtually all tight junctions in the oesophagus wall.

The composition according to the invention may comprise a viscosity controlling agent selected form acacia gum, tragacanth, alginic acid, carrageenan, locust bean gum, guar gum, gelatine, polyacrylate, methylcellulose, carboxymethylcellulose, xanthan gum, gellan gum, curdlan, dextran, pullulan, scleroglucan, or a combination thereof. This allows the composition to move slowly through the oesophagus and quercetin, myricetin, genistein, and/or berberine can more effectively reinforce the barrier strength of the oesophagus, and as a result reduce or eliminate heartburn, GERD and/or EoE symptoms.

The viscosity controlling agent in the composition according to the invention is preferably alginic acid. The viscosity of alginic acid is increased upon contact with stomach acid. The composition according to the invention comprising alginic acid can have an initial viscosity which is relatively low, allowing the composition to be easily swallowed. As this composition comes into contact with risen stomach acid in the oesophagus, the viscosity increases at the location of contact. In this contact area, the active components of the composition, quercetin, myricetin, genistein, and/or berberine thus reside for a longer time due to the locally increased viscosity of the composition. This further increases the effectiveness locally in the oesophagus.

Examples of synergetic compositions comprising quercetin (Q), myricetin (M), genistein (G), berberine (B), and/or acacia gum (AG) are: Q and AG; M and AG; Q, M, and AG; Q, G, and AG; Q, B, and AG; Q, M, G, and AG; Q, M, B, and AG; Q, M, G, B, and AG; M, G and AG, M, B and AG.

Examples of synergetic compositions comprising quercetin (Q), myricetin (M), genistein (G), berberine (B), and/or tragacanth (TC) are: Q and TC; M and TC; Q, M, and TC; Q, G, and TC; Q, B, and TC; Q, M, G, and TC; Q, M, B, and TC; Q, M, G, B, and TC; M, G and AG, M, B and TC.

Examples of synergetic compositions comprising quercetin (Q), myricetin (M), genistein (G), berberine (B), and/or alginic acid (AA) are: Q and AA; M and AA; Q, M, and AA; Q, G, and AA; Q, B, and AA; Q, M, G, and AA; Q, M, B, and AA; Q, M, G, B, and AA; M, G and AA, M, B and AA.

Examples of synergetic compositions comprising quercetin (Q), myricetin (M), genistein (G), berberine (B), and/or carrageenan (CG) are: Q and CG; M and CG; Q, M, and CG; Q, G, and CG; Q, B, and CG; Q, M, G, and CG; Q, M, B, and CG; Q, M, G, B, and CG; M, G and CG, M, B and CG.

Examples of synergetic compositions comprising quercetin (Q), myricetin (M), genistein (G), berberine (B), and/or locust bean gum (LB) are: Q and LB; M and LB; Q, M, and LB; Q, G, and LB; Q, B, and LB; Q, M, G, and LB; Q, M, B, and LB; Q, M, G, B, and LB; M, G and LB, M, B and LB.

Examples of synergetic compositions comprising quercetin (Q), myricetin (M), genistein (G), berberine (B), and/or guar gum (GG) are: Q and GG; M and GG; Q, M, and GG; Q, G, and GG; Q, B, and GG; Q, M, G, and GG; Q, M, B, and GG; Q, M, G, B, and GG; M, G and GG, M, B and GG.

Examples of synergetic compositions comprising quercetin (Q), myricetin (M), genistein (G), berberine (B), and/or gelatine (GL) are: Q and GL; M and GL; Q, M, and GL; Q, G, and GL; Q, B, and GL; Q, M, G, and GL; Q, M, B, and GL; Q, M, G, B, and GL; M, G and GL, M, B and GL.

Examples of synergetic compositions comprising quercetin (Q), myricetin (M), genistein (G), berberine (B), and/or polyacrylate (PA) are: Q and PA; M and PA; Q, M, and PA; Q, G, and PA; Q, B, and PA; Q, M, G, and PA; Q, M, B, and PA; Q, M, G, B, and PA; M, G and PA, M, B and PA.

Examples of synergetic compositions comprising quercetin (Q), myricetin (M), genistein (G), berberine (B), and/or methylcellulose (MC) are: Q and MC; M and MC; Q, M, and MC; Q, G, and MC; Q, B, and MC; Q, M, G, and MC; Q, M, B, and MC; Q, M, G, B, and MC; M, G and MC, M, B and MC.

Examples of synergetic compositions comprising quercetin (Q), myricetin (M), genistein (G), berberine (B), and/or carboxymethylcellulose (CM) are: Q and CM; M and CM; Q, M, and CM; Q, G, and CM; Q, B, and CM; Q, M, G, and CM; Q, M, B, and CM; Q, M, G, B, and CM; M, G and CM, M, B and CM.

Examples of synergetic compositions comprising quercetin (Q), myricetin (M), genistein (G), berberine (B), and/or xanthan gum (XG) are: Q and XG; M and XG; Q, M, and XG; Q, G, and XG; Q, B, and XG; Q, M, G, and XG; Q, M, B, and XG; Q, M, G, B, and XG; M, G and XG, M, B and XG.

Examples of synergetic compositions comprising quercetin (Q), myricetin (M), genistein (G), berberine (B), and/or gellan gum (EG) are: Q and EG; M and EG; Q, M, and EG; Q, G, and EG; Q, B, and EG; Q, M, G, and EG; Q, M, B, and EG; Q, M, G, B, and EG; M, G and EG, M, B and EG.

Examples of synergetic compositions comprising quercetin (Q), myricetin (M), genistein (G), berberine (B), and/or curdlan (CU) are: Q and CU; M and CU; Q, M, and CU; Q, G, and CU; Q, B, and CU; Q, M, G, and CU; Q, M, B, and CU; Q, M, G, B, and CU; M, G and CU, M, B and CU.

Examples of synergetic compositions comprising quercetin (Q), myricetin (M), genistein (G), berberine (B), and/or dextran (DX) are: Q and DX; M and DX; Q, M, and DX; Q, G, and DX; Q, B, and DX; Q, M, G, and DX; Q, M, B, and DX; Q, M, G, B, and DX; M, G and DX, M, B and DX.

Examples of synergetic compositions comprising quercetin (Q), myricetin (M), genistein (G), berberine (B), and/or pullulan (PU) are: Q and PU; M and PU; Q, M, and PU; Q, G, and PU; Q, B, and PU; Q, M, G, and PU; Q, M, B, and PU; Q, M, G, B, and PU; M, G and PU, M, B and PU.

Examples of synergetic compositions comprising quercetin (Q), myricetin (M), genistein (G), berberine (B), and/or scleroglucan (SC) are: Q and SC; M and SC; Q, M, and SC; Q, G, and SC; Q, B, and SC; Q, M, G, and SC; Q, M, B, and SC; Q, M, G, B, and SC; M, G and SC, M, B and SC

The viscosity controlling agent in the composition according to the invention may be present in an amount ranging from 0.05-10 wt. %, based on the total weight of the composition. Such an amount of viscosity inducing agent in the composition allows the composition to be sufficiently viscous to effectively increase the residence time in the oesophagus and to be sufficiently fluid to be easily swallowed.

The composition according to the invention may comprise an iron containing compound selected from ferrous sulfate, ferrous gluconate, iron bisglicinate, ferrous salts, ferric salts, iron polysaccharide complex, iron and ascorbic acid complex, heme iron, elemental iron, ferrous fumarate, ferronyl iron, Fe²⁺, Fe³⁺, or combinations thereof. In addition to the iron containing compound, the composition according to the invention may comprise vitamins and micronutrients in a dosage required during pregnancy. Pregnant women in particular suffer from heartburn and GERD, and are recommended to ensure complete vitamin and micronutrient intake plus require about twice the amount of iron because of the formation of additional blood for the baby. In addition, acid reflux can cause iron deficiency. When the composition according to the invention contains alginate, iron cations derived from the iron containing compound can interact with carboxylic groups in the alginate backbone to form ionic crosslinks, and as such, form a hydrogel having a high viscosity. This increased viscosity allows the composition to effectively treat heartburn, GERD and/or EoE symptoms locally in the oesophagus. The composition according to the present invention preferably does not comprise a hydrolyzed protein source comprising whey and casein; and the composition according to the present invention preferably does not comprise a lipid; at least one pre-gelatinized starch; a low-methylated pectin; and/or at least one additional carbohydrate (but optionally any of these may be comprised in the composition).

The composition according to the invention may be for administration to a paediatric patient, i.e. patient with age 0-18 years, or 0-12 years, or to a pregnant woman. Hence, the composition according to the invention may comprise nutritional supplementation to help safeguard that all nutritional needs are met during pregnancy. Pregnant women are commonly recommended such supplementation because of heightened nutritional needs combined with digestive troubles caused by the pregnancy, including nausea/vomiting, constipation and heartburn complaints.

The composition according to the invention may comprise quercetin and/or myricetin, both of which modulate Par6, and genistein or berberine, which reinforce tight junctions, as quercetin and myricetin do, but which do not modulate Par6. This composition preferably is a plant-protein emulsion wherein quercetin and/or myricetin and genistein or berberine are suspended. This emulsion may contain alginic acid or a salt thereof in a concentration of 0.1-5 wt. %, 0.2-1 wt. %, or 0.2-0.5 wt. %, optionally supplemented with vitamins and/or iron containing compounds. Also, a flavouring agent may be comprised in the composition according to the present invention, for example mint. An additional benefit is that this may stimulate saliva production.

### BRIEF DESCRIPTION OF THE FIGURES

FIG. 1 is a schematic drawing of the functioning of the oesophagus and the effect of the composition according to the invention.

FIG. 2 is a graph of activity data for compounds Myricetin and Quercetin.

### EXAMPLES

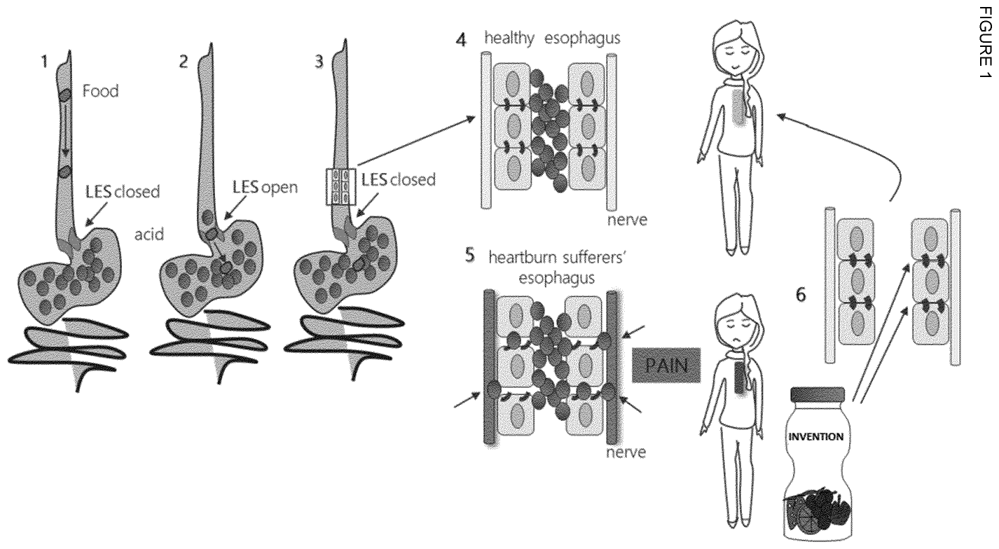

Referring now to FIG. 1. When food is swallowed and travels down the oesophagus (1), the lower esophageal sphincter (LES) opens, allowing food to enter the stomach and a little bit of acid travels upwards into the oesophagus (2). Subsequently, the LES closes to prevent food and more acid from flowing back (3). In a healthy oesophagus, acid is contained because cells are tightened together (4). Patients with heartburn and GERD have an acid-sensitive oesophagus. Cells are spaced apart and stomach acid can reach the underlying nerves, causing pain (5). The composition according to the invention effectively reinforces the oesophagus wall, restoring oesophagus health (6), and reducing or eliminating heartburn and GERD symptoms.

The biological barriers of concern in heartburn, GERD and EoE are epithelia, which consist of epithelial cells. Epithelia are oesophagus epithelium and connections between the epithelial cells that seal the space between cells, tight junctions (TJs), see FIG. 1.

### Active Compounds

The present inventors used a unique approach in order to identify compounds which reinforce the TJ system via the control of TJ initiation. Par6 was selected as the biological target; it acts as a regulator of tight junction initiation. Par6 has a polar distribution, with a defined zone of high abundance at one end of the cell. This Par6 distribution is key to the biological function, not just the absolute Par6 protein level.

Standard tools for RNA and protein level measurement are not adequate to discover compounds which target Par6 and the present inventors devised and implemented an assay where the distribution of Par6 in living tissues can be monitored: Thelial Live Targeted Epithelia (theLiTE™; U.S. Pat. No. 9,416,391).

The technology of theLITE™ allows monitoring the distribution of Par6—an intracellular protein that controls tight junction assembly. The assay was performed in live follicular epithelia of the egg chamber of the fruit fly Drosophila. Par6 was tagged with a green fluorescence protein (GFP) and its distribution within the cell was monitored by the GFP signal. In barrier cells Par6 has a polar distribution, with a defined zone of high abundance for example at one end of the cell. This protein distribution is key to the biological function, not just the absolute protein level. Standard tools for RNA and protein level measurement are not adequate because these techniques do not inform about the distribution of Par6 within the cell.

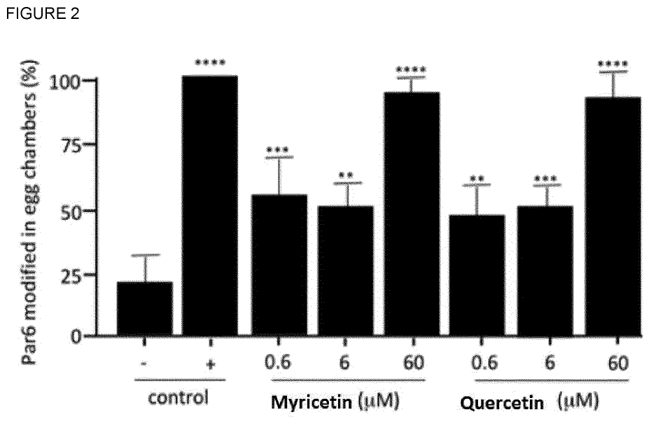

In theLiTE, the present inventors tested whether compounds modulate the distribution of Par6 protein in live follicular epithelia. In theLiTE more than 20 live egg chambers were exposed to test each compound and monitored for Par6 distribution after a set incubation time; any positive compounds were tested in three separate runs of experiments and over a broad range of concentrations. The full method is disclosed in the US patent document. In total over 2000 individual compounds were screened; four candidates with potent Par6 modulation and low toxicity were short-listed. Two, Myricetin and Quercetin, are natural flavonoid compounds either or both of which can be formulated in the composition according to the present disclosure. The activity data for compounds Myricetin and Quercetin are shown in FIG. 2. The positive control in FIG. 2, indicated with a “+” sign, is Antimycin A, at a concentration of 40 micromolar. The negative control, indicated with a “−” sign is the solvent medium (Insect cell culture medium plus 10% dimethyl sulfoxide). Antimycin A (CAS number 1397-94-0) is toxic, and therefore not useful in therapy. Nevertheless, it was identified as the most potent compound in the test and as such included as a positive control.

Myricetin and Quercetin are widely known and well characterized flavonoid compounds. Name and industry standard identifications are listed in Table 1.

TABLE 1
Quercetin and myricetin data

| Name: | Quercetin | Myricetin |
|---|---|---|
| CAS | 117-39-5 | 529-44-2 |
| Number: | | |
| IUPAC | 2-(3,4-dihydroxyphenyl)-3,5,7- | 3,5,7-Trihydroxy-2-(3,4,5- |
| Name: | trihydroxychromen-4-one | trihydroxyphenyl)-4H-1- |
| | | benzopyran-4-one |

### Preclinical Data—Cell Culture

The industry standard assays for measuring barrier strength and reinforcement in vitro are based on cell culture. Briefly, cells are cultured on a permeable substrate in a two-chamber device until they form a complete barrier layer, separating the two chambers. Fluorescent dye Lucifer yellow is added to one chamber and passage through the cell monolayer to the second chamber monitored. The lower the dye efflux, the stronger the barrier.

Quercetin is reported to have similar barrier reinforcing effects in Caco-2 and oesophagus derived cell line HET2A at a concentration of 20 to 50 micromolar (e.g. NIH project 5R21DK097529-02 accessed 19.02.2020 via http://grantome.com/grant/NI H/R21-DK097529-02).

### Preclinical Data—Animal Models

A variety of animal models which recapitulate barrier defects in oesophagus are available. However they are considered poor models for the clinical situation because of structural differences and the fact rodent oesophagus' tissue is keratinized, whilst the human oesophagus is not.

### Clinical Data

Clinical Trial—Patients with Non-Erosive GERD

A clinical trial of Quercetin in patients with non-erosive Gastro esophageal Reflux Disease (GERD) has been approved by the relevant ethical committees and is completed by team directed by Professor Roy C Orlando at the University of North Carolina at Chapel Hill. The trial is funded by the U.S. National Institutes of Health (NIH), Grant #R21-DK097529, and study description available at NIH's ClinicalTrials.gov website, identifier #NCT02226484. Summary: In this open-label trial subjects are all given 500 mg of Quercetin orally twice daily (tablet form) 1 hour before the morning and the evening meal for 6 weeks. Endoscopic esophageal biopsies are scheduled before (day 0) and after (day 56) Quercetin therapy for evaluation of esophageal epithelial barrier function. Quercetin increases esophageal epithelial barrier function, and specifically its resistance to injury upon contact with hydrochloric (gastric) acid (Clinicaltrials.gov NCT02226484, unpublished).

### Efficacy in a Commercially Relevant Population

27 pregnant women self-identifying as suffering from heartburn symptoms volunteered and were provided with a monthly supply of a lozenge format of the invention disclosed here (a 2,5 g isomalt lozenge comprising 38 mg Quercetin as active ingredient plus selected B vitamins), to be consumed daily through dispersion by sucking in the mouth for 30 consecutive days. Participants self-report symptom improvement upon completion in 70 percent of cases. Of the participants who experienced relief, 62% reported near immediate relief (15 to 30 mins after usage) strongly consistent with a localized effect of the invention disclosed here. By comparison, 14 GERD sufferers in clinical trial NCT02226484 who were administered twice 500 mg quercetin daily for a 6-week period were monitored for heartburn symptom improvement. There is no report of consistent effects.

10 persons diagnosed with EoE are provided with a monthly supply of a lozenge format of the invention disclosed here (a 2,5 g isomalt lozenge comprising 38 mg Quercetin as active ingredient plus selected B vitamins), to be consumed daily after breakfast through dispersion by sucking in the mouth for 30 consecutive days. Participants self-report symptom improvement including reduced food sensitivities upon completion in 70 percent of cases.

### Synergy Between Active Compounds

In a 6-weeks treatment, 50 subjects suffering from GERD receive a twice daily oral dose (1 hour before the morning and the evening meal) with either
Quercetin (500 mg);
Myricetin (500 mg);
Quercetin (500 mg) and Myricetin (500 mg);
Quercetin (500 mg) and Genistein (300 mg);
Myricetin (500 mg) and Berberine (750 mg).

| No. of | | |
|---|---|---|
| GERD | | |
| patients | Treatment | Observed Effect |
| 14 | Quercetin | 12 subjects report reduction in severity of GERD, |
| | | as well as reduced usage of PPIs |
| 14 | Myricetin | 10 subjects report reduction in severity of GERD, |
| | | as well as reduced usage of PPIs |
| 8 | Quercetin + | 8 subjects report suppression of GERD symptoms |
| | Myricetin | and a near complete elimination of the need to |
| | | use PPIs and antacids, for limited period of time |
| | | (~4 hours) |
| 7 | Quercetin + | 6 subjects report that GERD is resolved for |
| | Genistein | limited period of time (~4 hours) |
| 7 | Myricetin + | 5 subjects report that GERD is resolved for |
| | Berberine | limited period of time (~4 hours) |

The use of Quercetin alone or Myricetin alone leads to a reduction of GERD severity and a reduction in the need to use PPIs to resolve remaining symptoms. The use of the combination of Quercetin and Myricetin leads to suppression of GERD symptoms and a near complete elimination of the need to use PPIs and antacids.

The use of the combination of Quercetin and Genistein, as well as the combination of Myricetin and Berberine show further synergistic effects for completely resolving GERD. The present inventors consider that this may be due to their complementing mechanisms of action. Myricetin and Quercetin both act through modulating Par6, while Genistein and

Berberine are examples of flavonoids which reinforces tight junctions but not through modulating Par6 (data not shown). Having two molecules which both reinforce the epithelial barrier but acting via different routes leads to considerable synergy.

### Formulation

The prototype formulation used in the first clinical trial was a tablet. As with all solid foods, it typically passes through the oesophagus in under 10 seconds, too rapidly for the active compounds to have their full effect.

Usage of a dissolve in the mouth tablet, such as 2,5 g compressed lozenge comprised of isomalt plus active ingredients allows a prolonged, controlled and localized exposure of the oesophagus tissue to the active ingredient; consumption of such a lozenge by probands revealed times to complete dissolution in excess of five minutes.

1. A method for prevention or treatment of gastroesophageal reflux disease in a patient in need thereof comprising administering to the patient a composition comprising quercetin (2-(3,4-dihydroxyphenyl)-3,5,7-trihydroxychromen-4-one) and/or myricetin (3,5,7-trihydroxy-2-(3,4,5-trihydroxyphenyl)-4H-1-benzopyran-4-one), wherein the quercetin and/or myricetin is comprised in the composition in an amount of 0.01-100 mg, and wherein the composition does not comprise a hydrolyzed protein source comprising whey and casein; a lipid; at least one pre-gelatinized starch;
a low-methylated pectin; and/or at least one additional carbohydrate.

2. A method for prevention or treatment of Eosinophilic Esophagitis in a patient in need thereof comprising administering to the patient a composition comprising quercetin (2-(3,4-dihydroxyphenyl)-3,5,7-trihydroxychromen-4-one) and/or myricetin (3,5,7-trihydroxy-2-(3,4,5-trihydroxyphenyl)-4H-1-benzopyran-4-one).

3. The method according to claim 1, wherein the method reinforces oesophagus wall barrier function in the patient.

4. The method according to claim 1, wherein the patient is a pregnant woman or a paediatric patient.

5. The method for use according to claim 1, the composition comprising quercetin (2-(3,4-dihydroxyphenyl)-3,5,7-trihydroxychromen-4-one) and myricetin (3,5,7-trihydroxy-2-(3,4,5-trihydroxyphenyl)-4H benzopyran-4-one).

6. The method for use according to claim 1, wherein the composition comprises genistein (5,7-dihydro-3-(4-hydroxyphenyl)chromen-4-one) and/or berberine (5,6-dihydro-9,10-dimethoxybenzo[g]-1,3-benzodioxolo[5,6-a] quinolizinium).

7. The method for use according to claim 1, wherein an amount of quercetin ranges between 25 and 100 mg.

8. The method for use according to claim 1, wherein an amount of myricetin ranges between 25 and 50 mg.

9. The method for use according to claim 6, wherein an amount of genistein ranges between 0.01 and 600 mg.

10. The method for use according to claim 6, wherein an amount of berberine ranges between 0.01 and 1500 mg.

11. The method for use according to claim 1, wherein the composition is administered in a solid dosage form.

12. The method for use according to claim 1, wherein the composition is administered present in a liquid dosage form.

13. Composition for use according to claim 12, wherein the composition comprises a viscosity controlling agent selected from acacia gum, tragacanth, alginic acid, carrageenan, locust bean gum, guar gum, gelatine, polyacrylate, methylcellulose, carboxymethylcellulose, xanthan gum, gellan gum, curdlan, dextran, pullulan, scleroglucan, or a combination thereof

14. The method according to claim 13, wherein the viscosity controlling agent is alginic acid.

15. The method according to claim 13, wherein the viscosity controlling agent is present in an amount ranging from 0.05 — 10 wt. %, based on the total weight of the composition.

16. The method according to claim 14, wherein the viscosity controlling agent is present in an amount ranging from 0.05-10 wt. %, based on the total weight of the composition.

17. The method according to claim 2 wherein the method reinforces oesophagus wall barrier function in the patient.

18. The method according to claim 2, wherein the patient is a pregnant woman or a paediatric patient.

19. The method according to claim 2, the composition comprising quercetin (2-(3,4-dihydroxyphenyl)-3,5,7-trihydroxychromen-4-one) and myricetin (3,5,7-trihydroxy-2-(3,4,5-trihydroxyphenyl)-4H-1-benzopyran-4-one).

20. The method according to claim 2, wherein the composition comprises genistein (5,7-dihydro-3-(4-hydroxyphenyl)chromen-4-one) and/or berberine (5,6-dihydro-9,10-dimethoxybenzo[g]-1,3-benzodioxolo[5,6-a]quinolizinium).
